SEQUENCE LISTING (32 sequences)

SEQ ID NO: 1 - DNA, 19 bp, Artificial Sequence
  primer
agatttggacctgcgagcg

SEQ ID NO: 2 - DNA, 18 bp, Artificial Sequence
  primer
gaagccggggcaactcac

SEQ ID NO: 3 - DNA, 40 bp, Artificial Sequence
  primer
aagactcggcagcatctccatgggagttattygtgggttt

SEQ ID NO: 4 - DNA, 40 bp, Artificial Sequence
  primer
gcgatcgtcactgttctccaccccttccctcaccaataaa

SEQ ID NO: 5 - DNA, 40 bp, Artificial Sequence
  primer
aagactcggcagcatctccagyaatgggyatgttaggytt

SEQ ID NO: 6 - DNA, 40 bp, Artificial Sequence
  primer
gcgatcgtcactgttctccacccaccctattctcarcarc

SEQ ID NO: 7 - DNA, 45 bp, Artificial Sequence
  primer
aagactcggcagcatctccatttttygtttagtttygatgtaggt

SEQ ID NO: 8 - DNA, 45 bp, Artificial Sequence
  primer
gcgatcgtcactgttctccacacataaactttccttttcccctca

SEQ ID NO: 9 - DNA, 41 bp, Artificial Sequence
  primer
aagactcggcagcatctccagtaggtagtgtagatagygtg

SEQ ID NO: 10 - DNA, 42 bp, Artificial Sequence
  primer
gcgatcgtcactgttctccatatctacactacctrcctaatc

SEQ ID NO: 11 - DNA, 46 bp, Artificial Sequence
  primer
aagactcggcagcatctccatggggtatatgggyttttgaaattaa

SEQ ID NO: 12 - DNA, 44 bp, Artificial Sequence
  primer
gcgatcgtcactgttctccaaactccaaactattcccttctcca

SEQ ID NO: 13 - DNA, 43 bp, Artificial Sequence
  primer
aagactcggcagcatctccatgygttyggttttattttaggaa

SEQ ID NO: 14 - DNA, 43 bp, Artificial Sequence
  primer
gcgatcgtcactgttctccatgygttyggttttattttaggaa

SEQ ID NO: 15 - DNA, 45 bp, Artificial Sequence
  primer
aagactcggcagcatctccaagtttaggattttagagygtgagtg

SEQ ID NO: 16 - DNA, 40 bp, Artificial Sequence
  primer
gcgatcgtcactgttctccaccccaaccaacaacraaacc

SEQ ID NO: 17 - DNA, 43 bp, Artificial Sequence
  primer
aagactcggcagcatctccaggggttgtgataygttgttttga

SEQ ID NO: 18 - DNA, 45 bp, Artificial Sequence
  primer
gcgatcgtcactgttctccaacrtctatcctaatcctaccactca

SEQ ID NO: 19 - DNA, 43 bp, Artificial Sequence
  primer
aagactcggcagcatctccatgytggttaagtgattttaggya

SEQ ID NO: 20 - DNA, 40 bp, Artificial Sequence
  primer
gcgatcgtcactgttctccactctaarctccaarcctrca

SEQ ID NO: 21 - DNA, 40 bp, Artificial Sequence
  primer
aagactcggcagcatctccaggaggtgatgygatggytta

SEQ ID NO: 22 - DNA, 40 bp, Artificial Sequence
  primer
gcgatcgtcactgttctccacccactrcctcactctcatc

SEQ ID NO: 23 - DNA, 43 bp, Artificial Sequence
  primer
aagactcggcagcatctccaagatggttttgggtaggagaaaa

SEQ ID NO: 24 - DNA, 40 bp, Artificial Sequence
  primer
gcgatcgtcactgttctccatacacctcccaaaacraccc

SEQ ID NO: 25 - DNA, 40 bp, Artificial Sequence
  primer
aagactcggcagcatctccaggygggggtaggtagtttty

SEQ ID NO: 26 - DNA, 40 bp, Artificial Sequence
  primer
gcgatcgtcactgttctccacacrcrctccaacaactaat

SEQ ID NO: 27 - DNA, 45 bp, Artificial Sequence
  primer
aagactcggcagcatctccaaggtgggatagtaaaattgagtaga

SEQ ID NO: 28 - DNA, 40 bp, Artificial Sequence
  primer
gcgatcgtcactgttctccaaacrccaacraacacacaac

SEQ ID NO: 29 - DNA, 41 bp, Artificial Sequence
  primer
aagactcggcagcatctccatgtgttttaagggtttggggt

SEQ ID NO: 30 - DNA, 43 bp, Artificial Sequence
  primer
gcgatcgtcactgttctccaacctttacacttttcatacrcct

SEQ ID NO: 31 - DNA, 47 bp, Artificial Sequence
  primer
aagactcggcagcatctccatggtttttagatgatttatgttggagt

SEQ ID NO: 32 - DNA, 41 bp, Artificial Sequence
  primer
gcgatcgtcactgttctccaacccaacacttaacttcacct

(19) United States
(12) Patent Application Publication
(10) Pub. No.: US 2024/0050536 A1
(43) Pub. Date: Feb. 15, 2024

(54) USE OF DEOXYRIBONUCLEASE I IN THE TREATMENT OF ENDOMETRIOSIS IN PATIENTS WITH AN EPIGENETIC SIGNATURE OF ENDOMETRIOSIS ON CELL-FREE DNA

(71) Applicant: Endolife, Paris (FR)

(72) Inventors: André HAZOUT, Paris (FR); Moncef BEN KHALIFA, Le Mesnil-Saint-Denis (FR)

(21) Appl. No.: 18/260,421

(22) PCT Filed: Jan. 5, 2022
(86) PCT No.: PCT/EP2022/050171

§ 371 Date: Jul. 5, 2023

(30) Foreign Application Priority Data
Jan. 6, 2021 (EP) 21305014.9

(51) Int. Cl. A61K 38/46; C12Q 1/6883; A61P 15/02

(52) U.S. Cl. CPC A61K 38/465; C12Q 1/6883; A61P 15/02; C12Y 301/21001; C12Q 2600/154; C12Q 2600/106

(57) ABSTRACT

The invention relates to a method for treating endometriosis, by administering DNase I, and, in particular, for treating a patient with a cell-free DNA level above normal, a fortiori if this cell-free DNA has a methylation profile typical of endometriosis.

### FIELD OF THE INVENTION

The present invention relates to the field of the treatment of endometriosis.

More particularly, the invention proposes a treatment by deoxyribonuclease I (DNase I), hydrolysing the excess free deoxyribonucleic acids (cell-free DNA), in patients suffering from chronic endometriosis pain and having a certain hyper- and hypomethylated gene profile.

### TECHNOLOGICAL BACKGROUND

Endometriosis

Endometriosis is a chronic inflammatory illness, oestrogeno-dependent, due to the migration of cells of the endometrium outside the uterine cavity, mainly in the pelvic organs and tissues. This inflammatory state associated with pelvic pain and, sometimes, with a state of infertility, afflicts 3 to 10% of young women of procreation age.

The illness is heterogeneous, ranging from superficial peritoneal and serous lesions to endometriosis cysts in the ovaries (endometrium) and nodules in the deep endometrium, and may often be accompanied by fibrosis and adherences. Endometriosis may occur in an prepubescent girl suffering pain (Marsh and Laufer 2005) and then from puberty to menopause, where the menstrual cyclicity and the pain disappear.

We know that the growth of ectopic endometrial cells is dependent on oestrogens. Endometriosis is widely described as a regurgitation of the menstrual blood with a migration of the cells from the endometrium to the neighbouring organs and beyond: abdomen, lungs, liver and brain.

Very rarely, endometriosis has also been found in the genitourinary tract in men (Rei et Feloney, 2018). Twenty-two cases of hepatic endometrioses have also been published (Liu et al., 2015). This is an argument against the predominant theory of retrograde menstrual flow. Only 10% of women develop endometriosis whereas menstrual regurgitation occurs in 76% to 90% of women of procreation age (Blumenkrantz et al., 1981; Halme et al., 1984).

As stated above, endometriosis is an illness characterised by the presence of ectopic endometrial cells. In the superficial forms, the less serious, these cells migrate and fix on the peritoneum. In the more severe forms, the cells gain various organs: the ovary, the bladder or the intestinal wall. This abnormal location is associated with severe inflammation. It causes pain, in particular during periods, and sometimes infertility. Surgery is then often necessary for eliminating the nodules.

Women suffering from a profound form of the illness have major oxidative stress in the peritoneum. This dress is accompanied by a strong increase in fragments of free DNA in the circulating blood (Zachariah et al 2004).

At this stage it is difficult to state whether endometriosis is the cause of the stress or whether it is the converse. However, experiments conducted in vitro show that inhibiting this oxidative stress blocks the proliferation of endometrial cells (Filev et al 2019).

40% to 90% of women affected by endometriosis suffer pain. The major symptom is recurrent pelvic pain, sometimes very acute, in particular during periods. The lesions are in fact sensitive to oestrogens and will therefore proliferate, bleed and leave fibrous scars at each menstrual cycle. Outside the time periods, the patients may also suffer during sexual relations (dyspareunia) or when they urinate or have a bowel movement (McPeak et al. 2018).

The illness may, rarely, be completely asymptomatic. In this case, it is discovered fortuitously when the patient consults because of infertility. The scientific explanation of this link is not entirely elucidated. Recent studies show moreover that, in these patients, the endometrium has hormonal profiles and the expression of abnormal genes (Naqvi et al 2014; Laganà et al 2019; Vassilopoulou et al 2019).

However, whereas clinicians estimate that 90% of women have retrograde bleeding, only 10% of them develop endometriosis lesions confirmed by laparoscopy.

Endometriosis not only has physical effects but also psychological, causing depression and anxiety and compromising social relations. In addition, endometriosis has a negative impact on the sex life and social relations (Della Corte et al 2020).

Diagnosis of Endometriosis

Diagnosis of endometriosis is made during laparoscopy and confirmed by analysis of the lesions extracted. Treatment is often medical for the slight to moderate forms (stages I and II in accordance with the revised AFS score, or ASRM score) and surgical immediately or after hormone therapy for the severe forms (stages III and IV in accordance with the revised AFS score, or ASRM score—American Society of Reproductive Medicine).

According to another classification, resulting from the Recommendations for Clinical Endometriosis Practice (RPC endometriosis) published by the High Health Authority and the National College of Gynaecologists and Obstetricians of France (CNGOF) in 2018, there are 3 types of endometriosis:
superficial (or peritoneal) endometriosis, which designates the presence of ectopic endometrial implants located on the surface of the peritoneum,
ovarian endometriosis: ovarian endometriosis is an ovarian cyst characterised by its chocolate-coloured liquid content, and
deep pelvic (or sub-peritoneal) endometriosis corresponds to lesions that infiltrate in depth to more than 5 mm under the surface of the peritoneum. Deep endometriosis can affect typically the uterosacral ligaments (50% of cases), the posterior vaginal fornix (15%), the intestine (20-25%), represented mainly by the anterior face of the rectum and the rectosigmoid junction, the bladder (10%), the ureters (3%) and, beyond the pelvic cavity, the sigmoid, the ascending colon, the appendix and the terminal ileum for the most frequent locations.

Endometriosis has been described alternatively as a hormonal or immune illness, and a genetic illness triggered by exposure to environmental factors. In addition, several studies suggest various epigenetic aberrations in the pathogenesis of endometriosis (Koninck et al. 2019).

The heterogeneity of the phenotype of the illness is authenticated by a large number of falsely negative laparoscopies in symptomatic women. Thus anatomopathological, immunohistochemical and epigenetic examinations of the lesions have not proved to be reliable (Ahn et al., 2017), in particular with regard to methylation of the genome DNA of the B receptor of progesterone, of e-cadherin, of homeobox A10 (HOXA10), of the oestrogen receptor beta, of the steroidogenic factor 1 (SF1) and of aromatase.

The aberrant expression of DNA methyltransferase, which adds a methyl group in position 5 of the cytosine bases in the CpG island (cytosine phosphate guanine) of the gene promoter, rendering silent the corresponding gene expression, has been demonstrated in endometriosis (Nasu et al, 2011).

Treatment of Endometriosis

When a patient is algesic, she is usually offered, as the first-line treatment, a hormonal treatment intended to suppress the periods (continuous single-phase oestroprogestative or progestative contraceptives, Danazol or GnRH analogues). This treatment reduces the pain through its antigonadotrophic effect. In addition these treatments, in a period of genital activity, prevent the achievement of a parental plan.

Cell-Free DNA

In the human blood, the presence of free DNA circulating without cells (cfDNA) was reported in 1948 (Mandel and Metais in 1948). cfDNA has been studied in a wide range of physiological and pathological conditions, in particular inflammatory illnesses, oxidative stress, infertility in a couple (EP2879696 B1) and malign tumours.

In healthy individuals, during phagocytosis, the apoptotic or necrotic bodies are ingested by the macrophages. Thus the phenomenon of release of free DNA cannot take place. On the other hand, when fragments of DNA remain in the nucleosomes that are released by the macrophages, they are protected from enzymatic degradation and thus remain in the blood circulation.

cfDNA is composed of double-strand nucleic acids with a lower molecular weight than genomic DNA. The size of these genomic fragments is variable, ranging with the shortest from 70 to 200 bp and with the longest up to 21 kb. cfDNA is present in the healthy subject at blood concentrations evaluated between 50 et 250 ng/ml (EP2879696 B1).

The biological mechanisms of release of free DNA into the blood are not fully known. Fragments of free DNA may come from necrotic cells swallowed up by the macrophages, which are then partially released. According to this hypothesis, the levels of cfDNA should be correlated with the extent of cell necrosis and/or apoptosis (Grabushnig et al 2020).

The clearance of cfDNA from the blood circulation is rapid (half life: 16.3 min.); cfDNA is sensitive to plasmatic nucleases, but renal and hepatic clearances also participate in elimination thereof.

cfDNA can be isolated from plasma and serum, but serum contains a concentration of DNA approximately 6 times greater (incorporation in the cells).

The levels of DNA and the fragmentation schemes offer interesting possibilities for diagnostic, prognostic and therapeutic purposes.

A study by Zachariah (2009) showed significantly higher nuclear and mitochondrial cfDNA in a group of women suffering from minimal to slight endometriosis than in a reference group (p=0.046). The threshold as from a ROC curve showed a70% sensitivity and an 87% specificity. The author concluded that circulating cfDNA could constitute a potential biomarker of minimal and mild endometriosis.

Endonuclease I (DNase I)

DNase I is the enzyme responsible for digestion of extracellular DNA.

It catalyses the deoxyribonucleic acids into nucleotides or polynucleotides.

It hydrolyses the phosphodiester bonds and makes a nick, preferably on the pyramidal bases. In the presence of manganese ions is cuts the two strands in blunt ends. With magnesium ions, it cuts a strand into small fragments.

Its abilities to hydrolyse DNA are frequently exploited in biochemistry, for example to detect the print of proteins fixed on a cell genome. In medicine, the human recombinant form of DNase I is, under the name Pulmozyme®, the most powerful mucolytic used in the continuous daily treatment of mucoviscidose.

The data shown in the patent EP2879696 B1, relating to the use of DNase I in women having infertility without identified cause (in particular without the diagnosis of endometriosis), has shown that an injection of 2500 UI of DNase 1, over a short period, reduced the level of circulating free DNA by 40%.

In the present time, there does not exist any study showing the efficacy of DNase I on the pain and inflammation of moderate to severe endometriosis.

### SUMMARY OF THE INVENTION

The present invention relates to the use of Dnase for treating endometriosis.

In particular, the present invention aims to reduce inflammation and pain in patients suffering from endometriosis with a high level of free DNA, a profile of candidate genes and a methylation differential that are all evocative, using an exogenous DNase I and maintaining or even favouring the possibility of a pregnancy.

### DETAILED DESCRIPTION

In a prior study, the inventors showed the pertinence of the level of cell-free DNA in diagnosing endometriosis, as well as an epigenetic profile of endometriosis (patent application PCT/EP2020/069015, not published before the filing of the present application).

This is because the inventors showed that: (i) a hypermethylation of the genes selected from CALD1, RRP1, FN1, FAM87B, TCEAL6, RPL29P2, ATP11A-AS1, DIP2C, SLCO2B1, RM12, MIR3170, LINC01007, TSPAN17, MIR4693, HYOU1, TLR4, ADGRL3, IL6, VIRMA, MKRN1, INSIG1, ROR2, MRPL3, FMNL2, TMEM19, ZNF438, LINC01192, RCBTB1, TSPAN33, NKD2, FGFR2, TPRG1, MIR4644, FOXO4, FSTL1 and CLMN, and/or (ii) a hypomethylation of the genes selected from STAU2-AS1, TDRD5, USP1, ACVR2A, FBXO38, NT5C2, NAV1, SOD3, C3, UBE3A, MIR4655, MYO5C, COX6C, MIR6133, BRSK2, MIR4277, MIR4251, MN1, MIR3666, AZIN1, MIR4251, SLC37A2, FZD10, FASN, MKRN9P and PCCA-AS1, constitute markers of endometriosis the development potential of which is the greater, the greater the hyper- or hypomethylation measured.

More particularly, the inventors identified a list of 15 genes highly involved in endometriosis:
(10 and 5), namely: CALD1, RRP1, FN1, FAM87B, TCEAL6, RPL29P2, ATP11A-AS1, DIP2C, SLCO2B1 and RM12, hyper-methylated in the cell-free DNA of women suffering from endometriosis, and
FBX038, ACVR2A, USP1, TDRD5 and STAU2-AS1, hypomethylated in the cell-free DNA of women suffering from endometriosis.

Continuing their research with a view to treating patients suffering from endometriosis, the inventors obtained the following experimental results, which provide proof that endometriosis can be treated by acting on the cell-free DNA of the patients, in particular by administering DNase to them.

Treatment of Endometriosis by Administering DNase

According to a first aspect, the present invention relates to the use of a DNase in the treatment of endometriosis.

“Treatment” means here any reduction or improvement in the gravity and/or progression of endometriosis, in particular the reduction of one or more symptoms thereof, such as for example a reduction in the inflammation and/or pain. This improvement may result from administering DNase alone, or from a therapy combining DNase and another therapy (for example surgery for the most serious forms).

According to a particular embodiment of the invention, the DNase administered is a recombinant DNase.

More particularly, the present invention can be implemented by administering DNase 1, such as dornase alfa (Pulmozyme®), currently indicated in the treatment of bronchial congestion for improving the respiratory function in patients suffering from mucoviscidosis.

Subgroups of Patients

In a particular embodiment of the invention, DNase is used in the treatment of endometriosis in a patient having a level of cell-free DNA statistically greater than the level of free DNA in women who do not have endometriosis.

In practice, a person skilled in the art can, by routine studies on cohorts of patients suffering from endometriosis and of controls (for example, women who do not have endometriosis), determine one or more threshold(s) of cell-free DNA (optionally different according to the type of endometriosis) from which treatment by DNase is particularly recommended. To determine the patients most liable to respond to the treatment, cell-free DNA can be measured from a sample of biological fluid of the patient. As biological fluid that can be used in the context of the invention, mention can be made non-limitatively of blood, plasma and serum.

The cfDNA can be quantified as indicated in the experimental part below, or by means of other methods described in the scientific literature, in particular fluorometric or spectrophotometric methods such as QUBIT® (Life Technologies) or NANODROP™ (Thermo Scientific). Recently, the analysis of cell-free DNA has been abundantly described in methods for diagnosing certain cancers or in the prenatal diagnosis of chromosome anomalies. Thus several technologies for isolating and analysing cell-free DNA have been described, both in scientific publications and in patent literature. A person skilled in the art can perfectly select, from the many technologies described, the one that seems to them appropriate for identifying the patients most in a position to benefit from a treatment by DNase.

According to another particular embodiment of the invention, DNase is used in the treatment of endometriosis in a patient the cell-free DNA of which has a particular profile; this is because, as mentioned above, the inventors have identified that hypermethylation of certain genes and/or hypomethylation in certain other genes, in cell-free DNA, constitutes a biomarker of endometriosis.

More particularly, the present invention relates to the use of Dnase for treating endometriosis in a patient the cell-free DNA of whom has a methylation profile such that:
(i) one or more genes selected in the group formed by the genes CALD1, RRP1, FN1, FAM87B, TCEAL6, RPL29P2, ATP11A-AS1, DIP2C, SLCO2B1 and RM12 is hypermethylated, and/or
(ii) one or more genes selected in the group formed by the genes FBX038, ACVR2A, USP1, TDRD5 and STAU2-AS1 is hypomethylated.

“Hypermethylation of a gene” (or “hypomethylation of a gene”) means here a methylation level higher than (or lower than) the normomethylation level of 15% of the coding sequence of the gene, causing an increase (or a suppression) of the expression of this gene.

Obviously, a person skilled in the art can enhance and/or refine the methylation profile of the cell-free DNA making it possible to identify the patients who would respond most favourably to a treatment according to the invention.

A person skilled in the art can in particular, by routine work, measure the methylation differential of all or some of the genes mentioned above (signature of 15 genes or also the list of 36 hypermethylated genes and 26 hypomethylated genes mentioned in the second paragraph of the detailed description) in various cohorts (patients suffering from endometriosis who have or have not responded to treatment by DNase) and thus establish, according to the technology used for measuring these methylation levels, reference profiles with which the profile of the patient will be compared. Where applicable, a person skilled in the art can establish profiles corresponding to various forms of endometriosis, by making these routine measurements in various cohorts of patients having more or less serious forms of endometriosis and who have, or have not, responded to treatment by DNase. The profile of the patient will then be compared with these various profiles, which will make it possible to specifically treat the patients liable to respond favourably to the treatment.

To identify the patients most in a position to respond to the treatment according to the invention, the methylation profile of their cell-free DNA can be measured by any method described in the scientific literature.

In particular, three major molecular methods, based on enzymatic, immunological or chemical detection, make it possible to map the methylated cytosines. In the context of the present invention, these various techniques can be combined with methods of hybridisation on chips or of high-rate sequencing for more detailed resolution. The four techniques most usually used are MeDIP-seq, WGBS, RRBS and 450K Bead Array. These various methods can easily be implemented by a person skilled in the art, thanks to the availability of detailed protocols in the literature, of commercial kits and of specialised laboratories. These various methods produce concordant results, with however variable sensitivities of detection of differentially methylated regions between samples. Thus, in the context of the present invention, the “normal” level of methylation for the genes analysed must be calibrated using the technique that will be used for measuring the methylation level of the genes using the biological sample.

Immunological Detection Method

The specific antibodies of the methylated cytosines allow detection by immunoprecipitation, in accordance with a method called MeDIP (Methylated DNA ImmunoPrecipitation). Conventionally, DNA is fragmented by sonication, and the most methylated fragments will be the most favourably precipitated in the presence of the antibody, making it possible to obtain a fraction of the genome enriched with methylation. In the context of the present invention, the sonication step is not essential, given the fragmented character of cell-free DNA. Coupled with the high-rate sequencing (MeDIP-seq), this method makes it possible to measure a local methylation density, with a resolution of approximately 200 nucleotides corresponding to the mean size of the fragments, at reasonable cost. It allows complete coverage of the gene, with however a bias for the regions most rich in CpG units.

Chemical Detection Methods

The only tool making it possible to interrogate the methylation status on the scale of the individual cytosine is based on bisulfite. In the presence of this chemical compound, the cytosines are converted into uracil, whereas the methylated cytosines are not affected. This method thus enables the methylation to be read by analysing the single nucleotide polymorphisms (SNPs), in which a T corresponds to a non-modified cytosine and a C to a methylated cytosine on the reference genome before conversion.

The complete sequencing of the DNA after treatment with bisulfite, or WGBS (whole-Genome Bisulfite Sequencing), makes it possible to access the methylation status of all the cytosines, representing the excellence of all the methods for mapping the genome methylation.

RRBS (Reduced Representation Bisulfite Sequencing) is a technique derived from WGBS, based on the prior selection of the genome regions rich in CpG by the use of restriction enzymes. By reducing the number of fragments to be sequenced, the cost and the depth of the sequencing are greatly improved thereby, in the same order of magnitude as MeDIP-seq.

Finally, the DNA converted with bisulfite can also be hybridised on an oligonucleotide chip, comprising specific oligonucleotides of the differentially methylated genes in the endometriosic patients.

Method of Administering DNase

When it is administered for treating bronchial congestion, this DNase is normally administered by inhalation of an aerosol produced by a compressed-air nebulisation system. However, this administration method does not make it possible—except possibly by increasing the doses administered—to obtain a sufficient plasma concentration to have a significant effect on the concentration of circulating cell-free DNA. Furthermore, the toxicological study of Pulmozyme® showed that this product did not have any secondary effects when it was administered by other methods, including intravenously. Thus, in the context of the present invention, DNase is preferentially administered intramuscularly. Naturally, a person skilled in the art can, on the basis of his general galenic knowledge, select any other administration method making it possible to obtain a concentration of DNase that is effective on a therapeutic level.

Posology and Therapeutic Protocols

In the experiments reported below, the inventors observed an appreciable clinical improvement in patients suffering from endometriosis treated for one month by intramuscular (IM) injection of DNase I, at a dose of 2500 UI every two days. In particular, several patients noted an improvement in their quality of life related to a reduction in pain and dyspareunia.

On a biological level, the inventors observed a significant reduction in levels of free DNA.

These results provide proof of the efficacy of DNase in the treatment of endometriosis, in particular in patients having high levels of cell-free DNA, and open the way to a novel way of treating this illness.

According to a particular embodiment of the invention, the DNase is administered for treating endometriosis at a dose of at least 2500 UI every two days, for at least two weeks, preferably at least three weeks, and even more preferably for one month or more.

According to another embodiment of the invention, a dose two times greater is administered to the patients. For example, a dose of 5000 UI can be administered every two days, or the DNase can be administered at a daily dose of 2500 UI.

According to another embodiment of the invention, a dose of 2500 UI every day or every two days is administered, until a significant improvement of the symptoms is observed (for example, until the pain has significantly decreased) and/or until the level of cell-free DNA has dropped below a predetermined threshold (corresponding, for example, to a level close to the level observed on average in subjects not suffering from endometriosis).

According to another embodiment of the invention, a dose of 2500 UI every day or every two days is administered for an initially determined period, for example for one, two or three months. The person skilled in the art will obviously be able to adjust this period according to the level of gravity of the endometriosis and/or other parameters such as, for example, the level of cell-free DNA in the patient at the start of treatment.

Naturally, a person skilled in the art is also capable, using his general knowledge in particular with regard to galenics, of identifying administration methods and/or prolonged-release galenic forms, making it possible to space apart the injections while keeping over time a satisfactory plasma concentration of DNase

Various therapeutic protocols are envisaged in the context of the present invention, dependent in particular on the level of seriousness of the endometriosis.

Thus, for the less serious stages (stage I or II endometriosis), the endometriosis can be treated solely by administering DNase as described above, while for the more severe forms (stage III or IV endometriosis), treatment by Dnase will advantageously be able to be supplemented by surgery.

According to another embodiment of the invention, the treatment includes a plurality of phases. In a first phase, referred to as “aggressive phase”, the patient will receive DNase at a fairly high dose, for example 2500 UI per day. This aggressive phase can have a fixed duration (for example from 1 to 3 months), or a duration determined by the clinical development of the illness or by a biological marker (such as the level of plasmatic cell-free DNA).

At the end of this aggressive phase, and in order to improve the result and/or to avoid a relapse, the treatment will be continued by a maintenance treatment, which will be selected by the practitioner according to various factors. For example, for a patient having a pregnancy plan, the treatment with DNase will be continued, where applicable by selecting a dose or a frequency that is lower than during the aggressive treatment. By way of example, an administration of 2500 UI once or twice per week can be envisaged, or even every two weeks or even once a month. For a patient not having a pregnancy plan, the maintenance treatment may be either continuation of the treatment by Dnase (under the same conditions as for a patient having a wish for pregnancy), or a hormone treatment intended to suppress periods (continuous single-phase oestroprogestative or progestative contraceptives, Danazol or GnRH analogues or antagonist).

The present invention is further illustrated in the following experimental part, which does not limit the scope thereof.

### EXAMPLES

Introduction

In a study on a group of 32 women (16 without endometriosis antecedent and 16 with endometriosis antecedents, treated medically and/or surgically), the inventors showed that on average the endometriosis group contained 56% more free DNA than the control group.

By comparing the cfDNA of 5 women in each of the two groups by a complete genome study (WGS) by analysing, for each of the genes identified, its methylation status, they next observed significant differences in the methylation level of certain genes.

This is because the inventors showed that: (i) a hypermethylation of the genes selected from CALD1, RRP1, FN1, FAM87B, TCEAL6, RPL29P2, ATP11A-AS1, DIP2C, SLCO2B1, RM12, MIR3170, LINC01007, TSPAN17, MIR4693, HYOU1, TLR4, ADGRL3, IL6, VIRMA, MKRN1, INSIG1, ROR2, MRPL3, FMNL2, TMEM19, ZNF438, LINC01192, RCBTB1, TSPAN33, NKD2, FGFR2, TPRG1, MIR4644, FOXO4, FSTL1 and CLMN, and/or (ii) a hypomethylation of the genes selected from STAU2-AS1, TDRD5, USP1, ACVR2A, FBXO38, NT5C2, NAV1, SOD3, C3, UBE3A, MIR4655, MYO5C, COX6C, MIR6133, BRSK2, MIR4277, MIR4251, MN1, MIR3666, AZIN1, MIR4251, SLC37A2, FZD10, FASN, MKRN9P and PCCA-AS1, constitute markers of endometriosis the development potential of which is the greater, the greater the hyper- or hypomethylation measured.

More particularly, the inventors identified a list of 15 genes highly involved in endometriosis (10 hypermethylated and 5 hypomethylated), namely: CALD1, RRP1, FN1, FAM87B, TCEAL6, RPL29P2, ATP11A-AS1, DIP2C, SLCO2B1, RM12, FBX038, ACVR2A, USP1, TDRD5 and STAU2-AS1.

These results were described in detail in the patent application PCT/EP2020/069015, not published before the filing of the present application.

Continuing their research with a view to treating patients suffering from endometriosis, the inventors obtained the following experimental results.

### Example 1: Clinical Improvement in Patients Suffering from Endometriosis Treated by DNase I

Materials and Methods

Selection of Patients
Nine young patients, aged from 26 to 39 years, algesic, primary infertile, whose endometriosis was formally attested to by laparoscopy, agreed to benefit for one month from treatment by exogenous DNase 1.
The patients all suffered from chronic abdominopelvic pains, dyspareunia and unwanted bleeding during their menstrual cycles.
All had benefited from laparoscopy to confirm the diagnosis of endometriosis.
They had strong stage II pelvic endometriosis at a minimum or more (Stage Ill and IV); schematically, stage IIA is located at the peritoneum and/or at the uterosacral ligaments or at the fallopian tubes; stage IIIA associates the ovaries and stage IV the digestive tract and in particular the rectovaginal septum.
The patients had had no current medical treatment for more than 3 months and should not take any analgesic during their treatment by DNase.
The women participating in the study were able to receive the information with regard to the treatment and to the monitoring of the treatment.
All were judged capable of evaluating their pain and communicating with the doctor for analysing their comfort of life.

Each of them signed a consent form after information about the nature and methods of treatment and accepted biological evaluation.

The Ethics Committee of the University of Sousse gave its agreement to the therapeutic project and to the bioclinical monitoring of the patients.

Treatment

An intramuscular (IM) injection of 1 ampoule of 2500 UI of DNase I (Pulmozyme; Laboratoire Roche France, Neuilly sur Seine) every two days for one month.

The solution for inhalation, Pulmozyme® (dornase alfa), is a sterile, clear, colourless and highly purified solution of human recombinant deoxyribonuclease I (rhDNase) that selectively cleaves DNA.

Pulmozyme® is normally administered by inhalation of an aerosol produced by a compressed-air nebulisation system. However, the systemic levels of rhDNase are very low (maximum 15%) after inhalation; it was therefore decided to administer the same product by IM injection in order to increase the concentration of DNase, having well noted that there were no secondary effects in the toxicological study of Pulmozyme, including intravenously.

The selection of 2500 UI every two days was dictated to us by the intention to lower the levels of free DNA to a level normally encountered in women without endometriosis or any other inflammatory pathology.

The period of one month of treatment appeared necessary to drastically reduce oxidative stress and inflammation.

Throughout the duration of the treatment, regular consultation made it possible to evaluate the intensity of the pain and the comfort of life of the patients.

Quality of Life: The Evaluation Instruments

Among the various questionnaires available, the patients were subjected to 8 items of PWI created in 2018 by Rush et al (Personal Well Being Index) relating to comfort of life, work achievements, social relations, safety, community communications, vision of the future, and overall life satisfaction, on a scale from 0 to 10 (from completely unsatisfactory to completely satisfactory).

To evaluate pain, the VAS scale (Visual Analogue Scale) was used for five components such as dysmenorrhea, dyspareunia, dyschesia, chronic pelvic pain and dysuria on a horizontal scale of 10 cm ranging from total absence of pain to unbearable pain.

Clinical Results

Influence on Pain
Seven of the nine patients treated noted a reduction in their pain. The other two were not able to be evaluated because of non-compliance with the procedure.
Six patients out of seven declared that they had a reduction in pain whatever the stage of endometriosis.

Five changed from 9 to 4 as a mark on the VAS scale and one, aged 38 years, stage Ill, from 8 to 4.

One patient, the youngest, aged 26 years, strong stage II endometriosis, did not have any significant reduction in pain.

It is known that surgical intervention on all types of endometriosis does not routinely eliminate pain. The latter also relates to the inflammatory context, bleeding of the ectopic endometrium centres and nervous activation or impaired activation of the nociceptive channels.

Inflammatory reaction is in fact accompanied by local secretion of cytokines (IL6, TNF alpha, NGF) and other prostaglandins (PGE2) responsible for uterine contractions, bleeding and pain.
Six eligible patients declared that they had had an improvement in their dyspareunia; marks from 8 to 4 on the PWI scale.

One of them declared total disappearance of dyspareunia and a spectacular improvement in their comfort of life: mark from 8 to 1.

Another, aged 38 years, stage III endometriosis, who experienced an improvement in pain of 50%, noted an improvement of only 10% for dyspareunia.

The influence of endometriosis on sexual relations and more generally on relationships is one of the essential data of this pathology.

No difference was observed dependent on the stage of endometriosis and the locations of the endometriosis centres.

For sexual dysfunctions, interpretation is made more tricky if account is taken of the psychological factors and other comorbidities.
Four patients out of seven habitually presenting with unwanted uterovaginal bleeding seven to ten days before their periods declared total disappearance thereof under treatment by Domase alfa.

This intrauterine effect underlies the same action at the ectopic sites also participating in the reduction of abdominopelvic pain. This result is very promising with regards to the efficacy of the treatment

### Example 2: Biological Effects of the Treatment by DNase I in Patients Suffering from Endometriosis

Materials and Methods

The analyses presented below were implemented by the ACOBIOM research platform at Montpellier.

Analysis of Methylation of DNA by Sequencing

The expression of certain genes can be controlled by methylation of the cytosines in the CpG di-nucleotide islands of DNA. Deregulation of methylation can cause various pathologies (neurological illnesses, cancer, etc). The method employed here is treatment by bisulfite, which converts the non-methylated cytosines into uracil. The methylated cytosines are protected and are thus easily identified by sequencing (Sanger method or platform of the NGS type (Next-Gen sequencing)).

Method for Extracting Serum Free DNA

We used the Qiagen QIAamp DNA Blood Mini Kit Cat No./ID: 51104 in accordance with the protocol «QIAamp DNA Mini and Blood Mini Handbook» version of 05/2016.

To 400 μl of serum pipetted in a 1.5 ml microtube, we added 40 μl of protease (Proteinase K) at a concentration of approximately 600 mAU/ml or 40 mAU/mg of protein and 400 μl of denaturation buffer containing guanidine (AL bufferfrom Qiagen) then mixed by vortex (5 times 5 seconds) before being incubated at 56° C. for 10 minutes.

To the previous mixture, 400 μl of 100% non-denatured ethanol was added and mixed by vortex (5 times 5 seconds). Half of this mixture (420 μl) was next loaded onto a column of the kit and centrifuged at 8000×g for 30 seconds. After having eliminated the previously centrifuged part, the other half (420 μl) was loaded onto the same column and centrifuged at 8000×g for 30 seconds.

The DNA fixed on the silica beads trapped in the Qiagen column was washed once with 750 μl of AW1 buffer (buffer containing guanidine and ethanol) and then centrifuged at 8000×g for 30 seconds. A second washing with 750 μl of AW2 buffer (buffer containing Tris, NaCl and ethanol) and centrifugation at 8000×g for 30 seconds.

The membrane trapping the DNA was dried by centrifugation for 1 minute at 14,000×g. Elution of the DNA was carried out in two stages with 25 μl of AE buffer (equivalent to the TE buffer Tris/EDTA 1 mM/0.1 mM) after incubation for 2 minutes each time and centrifugation at 8000×g for 1 minute each time.

The DNA was stored cold at between 0° C. and 8° C. or frozen at a temperature between −16° C. and −85° C.

Method for Measuring the Concentration of Free DNA

Equipment
Sequence of the primers used for amplifying a region of 86 base pairs spanning on exon 1 and intron 2 of the gene Homo sapiens ribonuclease P/MRP subunit p30, abbreviated as RPP30 (NM_006413, ENST00000371703.7): RNP30_F: AGATTTGGACCTGCGAGCG (SEQ ID NO: 1) and RNP30_R: GAAGCCGGGGCAACTCAC (SEQ ID NO: 2).
Real-time thermocycler type LightCycler 48011.
Master mix containing SYBRgreen Type LightCycler® 480 SYBR Green I Master (Cat. no 04707516001)

Method

A triplicate of 5 μl of DNA extract was added to 20 μl of 1× Master mix containing 0.5 μM of each primer. The amplification consists of an activation of 5 minutes at 95° C. and then 35 denaturation cycles at 95° C. for 10 seconds, hybridisation at 59° C. for 20 seconds, elongation at 72° C. for 15 seconds followed by a final elongation of 5 minutes at 72° C.

Calculation of the concentration is done by linear regression with a dilution scale of 10 times, in triplicate, of a human DNA of known concentration (starting from 500,000 copies/reaction to 1000 copies per reaction) and comparison of the Ct's (Cycle threshold) in accordance with the known methods described for quantitative PCR (qPCR). The length of the haploid human gene considered is 3.2×10⁹ base pairs with a molar mass of 650 g/mole/bp, which gives us 290 copies/ng of haploid DNA.

An example of results is presented in the table below:

TABLE

| Patient ID posting 1 | DNA concentration (ng/μl) |
|---|---|
| A | 4.1 |
| B | 1.4 |
| C | 4.2 |
| D | 1.7 |
| E | 5.2 |
| F | 0.5 |
| G | 2.8 |
| H | 0.4 |
| I | 1.9 |
| J | 0.3 |
| K | 2.5 |
| L | 4 |
| M | 0.6 |
| N | 3.1 |

Treatment of the DNAs Extracted with Bisulfite

The whole of the 45 μl of each of the samples is treated with bisulfite using the EZ DNA Methylation Kit of Zymo Research in accordance with the supplier protocol. The methylated cytosines will be protected and will not be converted, whereas the non-methylated cytosines will be converted into uracil (U). The elution volume at the end of the conversion procedure is 25 μl.

Monitoring of the Concentration of Treated DNAs

The concentration of the samples treated with bisulfite is measured using the Nano-Drop ND-1000 spectrophotometer of Thermo Scientific. 1 μl is used for making the measurement.

Example of results in the table below:

| | DNA | Absorbance | Absorbance | Quantity |
|---|---|---|---|---|
| Sample | concentration | ratio | ratio | of DNA |
| ID | (ng/μl) | 260/280 | 260/230 | in 20 μl |
| A | 10.57 | 1.4 | 0.98 | 211.4 |
| B | 9.23 | 1.26 | 0.58 | 184.6 |
| C | 24.16 | 1.49 | 1.05 | 483.2 |
| D | 8.31 | 1.32 | 0.72 | 166.2 |
| E | 16.42 | 1.6 | 1.21 | 328.4 |
| F | 5.84 | 1.03 | 0.65 | 116.8 |
| G | 6.78 | 1.61 | 0.77 | 135.6 |
| H | 3.91 | 1.36 | 0.54 | 78.2 |
| I | 6.73 | 1.19 | 0.67 | 134.6 |
| J | 4.04 | 0.99 | 0.39 | 80.8 |
| K | 11.44 | 1.37 | 0.33 | 228.8 |
| L | 14.45 | 2.11 | 1.76 | 289 |
| M | 6.4 | 1.23 | 0.88 | 128 |
| N | 6.92 | 1.49 | 0.87 | 138.4 |

PCR Reactions

Design of the PCR Primers

15 targets were identified during previous experiments (cf table below):

| feature.name | feature.desc | feature.id |
|---|---|---|
| CALD1 | caldesmon 1 | NM_033140 |
| RRP1 | ribosomal RNA processing 1 | NM_003683 |
| FN1 | fibronectin 1 | NM_212482 |
| FAM87B | family with sequence similarity 87 | NR_103536 |
| | member B | |
| TCEAL6 | transcription elongation factor A | NM_001006938 |
| | like 6 | |
| RPL29P2 | ribosomal protein L29 pseudogene 2 | NR_002778 |
| ATP11A-AS1 | ATP11A antisense RNA 1 | NR_046661 |
| DIP2C | disco interacting protein 2 homolog C | NM_014974 |
| SLCO2B1 | solute carrier organic anion | NM_007256 |
| | transporter family member 2B1 | |
| RMI2 | RecQ mediated genome instability 2 | NM_152308 |
| STAU2-AS1 | STAU2 antisense RNA 1 | NR_038406 |
| TDRD5 | tudor domain containing 5 | NM_001199091 |
| USP1 | ubiquitin specific peptidase 1 | NM_003368 |
| ACVR2A | activin A receptor type 2A | NM_001616 |
| FBXO38 | F-box protein 38 | NM_001271723 |

For each of the 15 targets a specific pair of PCR primers was defined. The modified bases are indicated in bold: Y (base T or C) and R (base A or G).

| | | | Size of |
|---|---|---|---|
| | SEQ ID | | amplicon |
| Primer | NO: | Sequence | (bp) |
| CALD1_F | 3 | AAGACTCGGCAGCATCTCCATGGGAGTTATTYGTGGGTTT | 179 |
| CALD1_R | 4 | GCGATCGTCACTGTTCTCCACCCCTTCCCTCACCAATAAA | |
| RRP1_F | 5 | AAGACTCGGCAGCATCTCCAGYAATGGGYATGTTAGGYTT | 201 |
| RRP1_R | 6 | GCGATCGTCACTGTTCTCCACCCACCCTATTCTCARCARC | |
| FN1_F | 7 | AAGACTCGGCAGCATCTCCATTTTTYGTTTAGTTTYGATGTAGGT | 223 |
| FN1-R | 8 | GCGATCGTCACTGTTCTCCACACATAAACTTTCCTTTTCCCCTCA | |
| FAM87B_F | 9 | AAGACTCGGCAGCATCTCCAGTAGGTAGTGTAGATAGYGTG | 236 |
| FAM87B-R | 10 | GCGATCGTCACTGTTCTCCATATCTACACTACCTRCCTAATC | |
| TCEAL6_F | 11 | AAGACTCGGCAGCATCTCCATGGGGTATATGGGYTTTTGAAATTAA | 220 |
| TCEAL6_R | 12 | GCGATCGTCACTGTTCTCCAAACTCCAAACTATTCCCTTCTCCA | |
| RPL29P2_F | 13 | AAGACTCGGCAGCATCTCCATGYGTTYGGTTTTATTTTAGGAA | 162 |
| RPL29P2_R | 14 | GCGATCGTCACTGTTCTCCATGYGTTYGGTTTTATTTTAGGAA | |
| ATP11A- | 15 | AAGACTCGGCAGCATCTCCAAGTTTAGGATTTTAGAGYGTGAGTG | 231 |
| AS1_F | | | |
| ATP11A- | 16 | GCGATCGTCACTGTTCTCCACCCCAACCAACAACRAAACC | |
| AS1_R | | | |
| DIP2C_F | 17 | AAGACTCGGCAGCATCTCCAGGGGTTGTGATAYGTTGTTTTGA | 202 |
| DIP2C_R | 18 | GCGATCGTCACTGTTCTCCAACRTCTATCCTAATCCTACCACTCA | |
| SLCO2B1_F | 19 | AAGACTCGGCAGCATCTCCATGYTGGTTAAGTGATTTTAGGYA | 229 |
| SLCO2B1_R | 20 | GCGATCGTCACTGTTCTCCACTCTAARCTCCAARCCTRCA | |
| RMI2_F | 21 | AAGACTCGGCAGCATCTCCAGGAGGTGATGYGATGGYTTA | 235 |
| RMI2_R | 22 | GCGATCGTCACTGTTCTCCACCCACTRCCTCACTCTCATC | |
| STAU2- | 23 | AAGACTCGGCAGCATCTCCAAGATGGTTTTGGGTAGGAGAAAA | 216 |
| AS1_F | | | |
| STAU2- | 24 | GCGATCGTCACTGTTCTCCATACACCTCCCAAAACRACCC | |
| AS1_R | | | |
| TDRD5_F | 25 | AAGACTCGGCAGCATCTCCAGGYGGGGGTAGGTAGTTTTY | 229 |
| TDRD5_R | 26 | GCGATCGTCACTGTTCTCCACACRCRCTCCAACAACTAAT | |
| USP1_F | 27 | AAGACTCGGCAGCATCTCCAAGGTGGGATAGTAAAATTGAGTAGA | 221 |
| USP1_R | 28 | GCGATCGTCACTGTTCTCCAAACRCCAACRAACACACAAC | |
| ACVR2A_F | 29 | AAGACTCGGCAGCATCTCCATGTGTTTTAAGGGTTTGGGGT | 180 |
| ACVR2A-R | 30 | GCGATCGTCACTGTTCTCCAACCTTTACACTTTTCATACRCCT | |
| FBXO38_F | 31 | AAGACTCGGCAGCATCTCCATGGTTTTTAGATGATTTATGTTGGAGT | 180 |
| FBXO38_R | 32 | GCGATCGTCACTGTTCTCCAACCCAACACTTAACTTCACCT | |

PCR Reaction Mixture

The PCR reactions are implemented in a final volume of 25 μl containing:

| | | | | |
|---|---|---|---|---|
| Taq’Ozyme HS mix 2X | 125 | μl | | |
| Primers Sense + Antisense 2 μM | 5 | μl | (0.4 | μM final) |
| H₂O pure | 6.5 | μl | | |
| DNA matrix treated with bisulfite | 1 | ng/μl | 1 | μl |

PCR Programme

| | | | |
|---|---|---|---|
| 95° | C. | 1 | minute |
| 95° | C. | 15 | seconds |
| 56° | C. | 15 | seconds |
| 72° | C. | 30 | seconds |
| ×45 | cycles | | |
| 72° | C. | 5 | minutes |

The PCR products generated are checked by electrophoresis in 1% agarose (3 μl) and are then sequenced. The cytosines that were initially methylated were “protected” and were not converted during the bisulfite treatment, whereas the non-methylated cytosines will be converted into uracil (U). Thus, on sequencing, a methylated cytosine will appear as a cytosine (C) whereas a non-methylated cytosine will appear as a thymine (T).

Biological Results

Influence of the Treatment on the Levels of Free DNA

Extraction of the serum free DNAs showed a reduction of 40% of the levels at the end of treatment, compared with the levels before treatment.

The very high free DNA levels of the 7 eligible patients before treatment were reduced by almost a half with 2500 UI of Domase I every two days. This suggests a dose effect and the possibility of further reducing the levels of circulating DNA by administering 2500 UI per day for better efficacy.

The Gene and Methylation Profile

The seven serum DNAs before treatment and the seven after treatment are subjected to a WGS (Whole Genome Sequencing), in order to determine in particular the methylation status of the candidate genes (10 hypermethylated and 5 hypomethylated) cited in the introduction of this experimental part.

Obtaining the results of this analysis has been delayed by the COVID-19 pandemic. Nevertheless, it is anticipated that the methylation profile of these genes will be substantially altered after treatment by Domase alfa, and the gene distribution, relatively unchanged, will reveal new data in the distribution.

The network study should reflect a new distribution of the metabolic channels testifying to the beneficial effect of the treatment.

### REFERENCE

Ahn S H, Singh V, Tayade C. Biomarkers in endometriosis: challenges and opportunities. Fertil Steril. 2017; 107(3):523-532.
Blumenkrantz M J, Gallagher N, Bashore R A, Tenckhoff H, 1981. Retrograde menstruation in women undergoing chronic peritoneal dialysis. Obstet Gynecol; 57:667-70.
Della Corte, Di Filippo C, Gabrielli O, Reppuccia S, La Rosa V L, Ragusa R, Fichera M, Commodari E, Bifulco G, Giampaolino P. The Burden of Endometriosis on Women's Lifespan: A Narrative Overview on Quality of Life and Psychosocial Wellbeing. Int. J. Environ. Res. Public Health 2020, 17, 4683.
Filev A D, Shmarina G V, Ershova E S, Veiko N N, Martynov A V, Borzikova M A, Poletkina A A, Dolgikh O A, Veiko V P, Bekker A A, Chirkov A V, Volynshchikov Z N, Deviataikina A S, Shashin D M, Puretskiy V K, Tabakov V J, Izhevskaya V L, Kutsev S I, Kostyuk S V, Umriukhin P E. Oxidized Cell-Free DNA Role in the Antioxidant Defense Mechanisms under Stress. Oxid Med Cell Longev. 2019; 8; 2019:1245749.
Grabuschnig S, Bronkhorst A J, Holdenrieder S, Rosales Rodriguez I, Schliep K P, Schwendenwein D, Ungerer V, Sensen C W. Putative Origins of Cell-Free DNA in Humans: A Review of Active and Passive Nucleic Acid Release Mechanisms.
Halme J, Hammond M G, Hulka J F, Raj S G, Talbert L M, 1984. Retrograde menstruation in healthy women and in patients with endometriosis. Obstet Gynecol; 64:151-4.
Hazout A, Montjean D, Cassuto N G, Belloc S, Dalleac A, Tesarik J and Benkhalifa M, 2018. Free Circulating Nucleic Acids and Infertility. JGWH.
Koninckx P R, Ussia A, Adamyan L, Wattiez A, Gomel V, Martin D C. Pathogenesis of endometriosis: the genetic/epigenetic theory. Fertil Steril. 2019; 111(2):327-340.
Laganà A S, Garzon S, Götte M, Viganò P, Franchi M, Ghezzi F, Martin D C. The Pathogenesis of Endometriosis: Molecular and Cell Biology Insights. Int J Mol Sci. 2019 Nov. 10; 20(22):5615.
Liu K, Zhang W, Liu S, Dong B, Liu Y, 2015. Hepatic endometriosis: a rare case and review of the literature. Eur J Med Res; 20:48.
Mandel P, Metais P, 1948. Blood plasma nucleic acids in humans. C R Session Soc Biol Ses Fil. 142: 241-243.
Marsh E, Marc R Laufer; Endometriosis in premenarcheal girls who do not have an associated obstructive anomaly. Fertil Steril. 2005 March; 83(3): 758-60.
McPeak A E, Allaire C, Williams C, Albert A, Lisonkova S, Yong P J. Pain catastrophizing and pain Health—Related Life Clin J Pain. 2018; 34(4):349-356.
Naqvi H, Ilagan Y, Krikun G, Taylor H S, 2014. Altered genome-wide methylation in endometriosis. Reprod Sci; 21:1237-43.
Nasu K, Kawano Y, Tsukamoto Y, Takano M, Takai N, Li H, Furukawa Y, Abe W, Moriyama M, Narahara H. J. Aberrant DNA methylation status of endometriosis: epigenetics as the pathogenesis, biomarker and therapeutic target. Obstet Gynaecol Res. 2011 July; 37(7): 683-95.
Rei C, Williams T, and M Feloney, 2018. Endometriosis in a Man as a Rare Source of Abdominal Pain: A Case Report and Review of the Literature, Case Reports in Obstetrics and Gynaecology, vol. 2018, Article ID 2083121.
Rush, G.; Misajon, R. Examining subjective wellbeing and health-related quality of life in women with endometriosis. Health Care Women Int. 2018, 39, 303-321.
Vassilopoulou L, Matalliotakis M, Zerviou M, Atalliotaki C, Krithinakis K, Matalliotakis I, Spandidos D, Goulielmos G. N. Defining the genetic profile of endometriosis. Review 2019. Experimental and Therapeutic Medicine.
Zachariah R, Schmid S, Radpour R, Buerki N, Xiu-Cheng Fan A, Hahn S, Holzgreve W, Zhong X Y, 2009. Circulating cell-free DNA as a potential biomarker for minimal and mild endometriosis. RBMonline: 407-411.

1. A method for treating endometriosis in a patient, comprising administering an effective dose of DNase to said patient.

2. The method according to claim 1, wherein said DNase is a recombinant DNase.

3. The method according to claim 1, wherein said DNase is DNase I.

4. The method according to claim 1, wherein said patient has a level of cell-free DNA statistically greater than the level of free DNA in women who do not have endometriosis.

5. The method according to claim 1, wherein cell-free DNA of the patient has a methylation profile such that:
(i) one or more genes selected from CALD1, RRP1, FN1, FAM87B, TCEAL6, RPL29P2, ATP11A-AS1, DIP2C, SLCO2B1 and/or RMI2 is hypermethylated, and/or
(ii) one or more genes selected from FBX038, ACVR2A, USP1, TDRD5 and/or STAU2-AS1 is hypomethylated.

6. The method according to claim 1, wherein the DNase is administered intramuscularly.

7. The method according to claim 1, wherein the DNase is administered at a dose of at least 2500 UI every two days, for at least two weeks.

8. The method according to claim 1, wherein the DNase is administered at a daily dose of 2500 UI.

9. The method according to claim 1, wherein the DNase is administered at a dose of 2500 UI every day or every two days, for 1 to 3 months.

10. The method according to claim 1, wherein the patient is suffering from stage I or II endometriosis.

11. The method according to claim 1, wherein the patient is suffering from stage III or IV endometriosis, in association with surgery.

12. The method according to claim 1, wherein, after a first aggressive treatment of a dose of at least 2500 UI every two days, for at least two weeks, the patient receives a maintenance treatment selected from hormone therapy and/or an administration of DNase at a lower dose or frequency than during the aggressive treatment.
